(19) United States
(12) Patent Application Publication
(10) Pub. No.: US 2006/0035873 A1
(43) Pub. Date: Feb. 16, 2006

(54) Methods for inducing apolipoprotein e secretion

(76) Inventors: Eric J Niesor, Nyon (CH); Ane Perez, Versoix (CH); Jean-Luc Thuillard, Cergue (CH); Craig L Bentzen, Bogis-Bossey (CH); Lan Mong Nguyen, Nyon (CH)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/381,111

(22) PCT Filed: Aug. 2, 2001
(86) PCT No.: PCT/EP01/08980

§ 371 Date: Jul. 22, 2005

(30) Foreign Application Priority Data
Aug. 4, 2000 (GB) 0019290.6

(51) Int. Cl. A61K 31/56; A61K 31/08; A61K 31/045; A61K 31/336

(52) U.S. Cl. 514/171; 514/475; 514/724; 514/722

(57) ABSTRACT

The invention provides a method for increasing apolipoprotein E (ApoE) in plasma and in tissues of a mammal by using a combination of an ApoE increasing amount of an activator of the orphan nuclear receptor FXR and an ApoE increasing amount of an activator of the orphan nuclear receptor LXRα. Also provided is the use of a combination of an ApoE increasing amount of a FXR activator and of an ApoE increasing amount of a LXRα activator, for the manufacture of a medicament for increasing ApoE in plasma and in tissues of a mammal.

The present invention relates to respectively activators of the orphan nuclear receptor FXR such as farnesol, chenodeoxycholic acid and activators of the orphan nuclear receptor LXRα such as 22(R)-hydroxycholesterol, pharmaceutical compositions containing them and their use in therapy for modulating and in particular for increasing apolipoprotein E in plasma and in tissues.

### BACKGROUND OF THE INVENTION

Apolipoprotein E (ApoE) is a polymorphic, multifunctional protein synthesized by several cell types and tissues, including liver, kidney, skin, adipose tissue, macrophages and brain. The wide distribution of ApoE is associated with the maintenance of key cellular functions such as intracellular cholesterol trafficking, cholesterol distribution between cells, and tissue reparation. The amino acid sequence of the ApoE protein is well conserved throughout species. ApoE can be viewed as a regulator of cholesterol homeostasis in tissues such as the Central Nervous System (CNS) and Peripheral Nervous System (PNS) and the arterial wall (cell-cell) or between tissues via the circulating plasma lipoproteins (tissue-tissue).

The major role of plasma ApoE containing lipoproteins is to transfer lipids (cholesterol) from peripheral tissues to the liver and to remove excess cholesterol from peripheral tissues via the reverse cholesterol transport system. Dysregulation of this mechanism leads to excess cholesterol deposition in peripheral tissues such as arteries (arterosclerosis) and skin (xanthomas and xanthelasmas). ApoE has also been shown to have a direct effect on lymphocyte proliferation and thus has an immunomodulatory role.

After the liver, the brain is the second major site of ApoE synthesis. ApoE is the only lipoprotein synthesized by brain tissue where the key role of ApoE is cholesterol transport between cells of the central nervous system (CNS). Local secretion of ApoE by cells such as macrophages or macrophage-derived cells is essential for the uptake of excess tissue cholesterol or provides cholesterol for specific needs such as nerve repair and remyelinisation.

Up to the present time, the compounds affecting Apo E production in vitro and in vivo have not been investigated thoroughly. Only hormone-like estrogens and corticoids have been shown to change Apo E levels under various experimental conditions; see for instance: Srivastava R A K, Srivastava N, Averna M, Lin R C, Korach K S, Lubahn D B, Schonfeld G “Estrogen up-regulates apolipoprotein E (ApoE) gene expression by increasing ApoE mRNA in the translating pool via the estrogen receptor alpha-mediated pathway” J Biol Chem, 1997, 272:33360-33366 and Stone D J, Rozovsky I, Morgan T E, Anderson C P, Hajian H, Finch C E “Astrocytes and microglia respond to estrogen with increased ApoE mRNA. in vivo and in vitro” Experimental Neurology, 1997, 143:313-318.

Orphan Nuclear Receptors FXR and LXR

Steroid hormones (glucocorticoids, mineralocorticoids, estrogens, progestins, androgens, and vitamin D) bind to their nuclear receptors which are transcription factors and by this means regulate expression of gene coding for specific proteins and control critical cellular activities; see for instance Meier, C., A. Journal of Receptor & Signal Transduction Research 1997, 17, 319-335. In the last ten years more than 100 mammalian genes have extended the family of steroid nuclear receptors and have been classified as orphan nuclear receptors for which ligands are unknown; see for instance Enmark, E. and Gustafsson, J. A. Molecular Endocrinology 1996, 10, 1293-1307.

The farnesoid X activated receptor (FXR; NR1H4) was identified in 1995 by Forman et al. Cell 1995, 81, 687-693. FXR functions as a heterodimer with the Retinoid X Receptor (RXR) and binds to the DNA via an inverted repeat element IR-1. FXR was originally described as activated by isoprenoids such as farnesol and juvenile hormone III. More recently, several investigators came to the conclusion that bile acids are the physiological ligands and activators of FXR; see for instance Makishima, M. et al Science 1999, 284, 1362-1365. Parks, D. J. et al Science 1999, 284, 1365-1368. Wang, H. B. et al Molecular Cell 1999, 3, 543-553.

The Liver X Receptor was identified as an orphan nuclear receptor by Willy et al Genes & Development 1995, 9, 1033-1045. and its ligand-binding domain shares 37% identity with FXR. The DNA binding sequence of LXR is a direct repeat separated by 4 nucleotides (DR-4) and differs from the DNA binding sequence of FXR (IR-1). Two genes encode for LXR proteins (LXRα;NR1H3 and LXRβ;NR1H2) and both receptors are activated by various oxysterols, the most potent being 22(R)-hydroxycholesterol, 24 (S)-hydroxycholesterol, 24(S),25-epoxycholesterol and 7-hydroxycholesterol; see for instance: Janowsky B. A. et al Proc Natl Acad Sci USA 1999, 96, 266-271. Thus LXRα appears to be a sensor of both isoprenoids and oxysterols but the exact physiological and pharmacological applications of the activators and ligands of any these receptors (FXR and LXR) are yet to be established (Niesor E et al Current Pharmaceutical Drug Design 2000 in press).

### SUMMARY OF THE INVENTION

The applicants have discovered that representative ligands and activators of FXR (e.g. the isoprenoid farnesol, the bile acid derivative chenodeoxycholic acid) and of LXRα (e.g. the hydroxysterol 22(R)-hydroxycholesterol) are potent inducers of ApoE secretion and might be useful in the treatment of diseases requiring an increased production of plasma and/or tissue ApoE.

Compounds which modulate ApoE synthesis and secretion should have application in the treatment of diseases such as
atherosclerosis,
excess lipid deposition in peripheral tissues such as skin (xanthomas),
stroke,
memory loss,
optic nerve and retinal pathologies (i.e. macular degeneration, retinitis pigmentosa),
repair of traumatic damage of the central nervous system (brain tissue),
repair of traumatic damage of the peripheral nervous system (i.e. nerve section compression or crush),
prevention of the degenerative process due to aging (i.e. Alzheimer's disease),
prevention of degenerative neuropathies occurring in diseases such as diabetic neuropathies and multiple sclerosis,
autoimmune diseases and
activation of the innate immune system.
   ApoE in Atherosclerosis

As a component of all lipoprotein fractions, ApoE plays a important role in cholesterol homeostasis, by mediating their interaction with receptors such as the apoB, low-density lipoprotein (LDL) and other specific receptors. The important role of ApoE in cardiovascular diseases is demonstrated by the ApoE knock-out mouse model where the animals rapidly develop hypercholesterolemia and atherosclerosis with pathological features similar to human atherosclerosis; see for instance: Plump A “Atherosclerosis and the mouse—a decade of experience [review]” Annals of Medicine 1997, 29:193-198.

In man, in the absence of a functional ApoE protein (human variants; see for instance: Richard P, Dezulueta M P, Beucler I, Degennes J L, Cassaigne A, Iron A “Identification of a new apolipoprotein E variant (E(2) Arg(142)- ]Leu) in type-III hyperlipidemia” Atherosclerosis. 1995, 112:19-28) and in the ApoE knock out mouse, plasma levels of cholesterol and triglycerides are abnormally high (even on a low fat diet) and atherosclerosis develops rapidly. In the animal model, these changes are prevented by infusion of ApoE, transplantation of macrophage producing ApoE or gene therapy by introducing the human ApoE gene into ApoE knock out mice, demonstrating the direct beneficial role of ApoE; see for instance: Linton M F, Atkinson J B, Fazio S “Prevention of atherosclerosis in apolipoprotein E-deficient mice by bone marrow transplantation” Science 1995, 267:1034-1037.

A recent study has examined the response of ApoE knock out mice to diets with increasing cholesterol content and has concluded that ApoE plays a critical role in decreasing the absorption of dietary cholesterol and in increasing biliary secretion, see for instance Sehayek E, Shefer S, Nguyen L B, Ono J G, Merkel M and Breslow J L,“Apolipoprotein E regulates dietary absorption and biliary cholesterol excretion: Studies in C57BL/6 ApoE knock out mice” Proc Natl Acad Sci USA 2000, 97, 3433-3437. The results of this study further suggest that increasing ApoE should decrease the absorption of dietary cholesterol and prevent the formation of biliary cholesterol stones.

ApoE in the Central Nervous System (CNS)

ApoE also plays a critical role in the central nervous system. In the brain ApoE is synthesized and secreted by astrocytes, its principal role being cholesterol transport between cells. ApoE is considered to redistribute lipids and to participate in the cholesterol homeostasis of the brain.

ApoE is linked to the neuropathological lesions characteristic of Alzheimer's disease with one isoform, ApoE4, strongly associated with the age of onset of the disease; (see for instance: Poirier J “Apolipoprotein E in animal models of CNS injury and in Alzheimer's disease” [review]Trends in Neurosciences 1994, 17:525-530 and Rubinsztein D C “Apolipoprotein E—a review of its roles in lipoprotein metabolism, neuronal growth and repair and as a risk factor for Alzheimer's disease” Psychological Medicine 1995, 25:223-229), while another isoform, ApoE3, is believed to help maintain healthy microtubules. In the brains of patients having Alzheimer's disease the observed increase in both ApoE mRNA and the number of astrocytes suggests that the ApoE increase represents an attempt of the astrocytes to repair the damage within the nervous cells.

In the absence of the ApoE gene (ApoE knock out mice) memory deficit, defect in the repair of brain injury and deposition of the Alzheimer's associated β-amyloid variant APP^{V717F} were demonstrated; see for instance: Oitzl M S, Mulder M, Lucassen P J, Havekes L M, Grootendorst J, Dekloet E R “Severe learaing deficits in apolipoprotein E knockout mice in a water maze task” Brain Research 1997, 752:189-196 and Laskowitz D T, Sheng H X, Bart R D, Joyner K A, Roses A D and Warner D S “Apolipoprotein E-deficient mice have increased susceptibility. to focal cerebral ischemia” Journal of Cerebral Blood Flow and Metabolism 1997, 17: 753-758 and Walker L C, Parker C A, Lipinski W J, Callahan M J, Carroll R T, Gandy S E, Smith J D, Jucker M and Bisgaier C L “Cerebral lipid deposition in aged Apolipoprotein E-deficient mice”, American Journal of Pathology 1997, 151:1371-1377.

Thus, increasing ApoE production in patients bearing the E2 and E3 isoforms of ApoE might have a beneficial effect on the occurrence of Alzheimer's or other spontaneous or traumatic neurological diseases.

ApoE in the Peripheral Nervous System (PNS)

The important role of ApoE in nerve regeneration in the peripheral nervous system is demonstrated by the observation that ApoE synthesis is dramatically induced when nerves are injured, see for intance: Poirier J 1994 “Apolipoprotein E in animal models of CNS injury and in Alzheimer's disease” [review], Trends in Neurosciences 1994, 17:525-530. The maintenance and/or repair of the myelin sheets involves the participation of ApoE secreted by support cells such as glial and Schwann cells. The ApoE synthesis and concentration were found to be abnormally low in degenerative diseases of nervous tissues such as in Multiple Sclerosis, see for instance: Gaillard O, Gervais A, Meillet D, Delattre J, Lyoncaen O, Schuller E “Apolipoprotein E intrathecal synthesis is decreased in multiple sclerosis patients” Annals of Clinical Biochemistry 1996, 33:2:148-150. ApoE is also considered to stabilize the cytoskeleton apparatus and support neurite elongation thus having a major effect on the development and remodelling following injury of the nervous system occurring late in life.

The above evidence has establislied the beneficial role of ApoE in lipid (cholesterol and triglyceride) homeostasis and also in nervous tissue homeostasis and recovery from injury. There is thus a rationale for the development of agents which effectively increase ApoE in plasma and tissues (liver, brain, central or peripheral nervous system) in order to treat lipid disorders such as atherosclerosis; or neurodegenerative disorders such as Alzheimer's disease or dementia and multiple sclerosis.

ApoE as Modulators of the Immune System

ApoE affects the immune system by acting on lymphocyte proliferation. Furthermore ApoE knock out mice are highly sensitive to bacterial infection due to a defect in their innate immune systems suggesting that increasing ApoE production should ameliorate the immune response; see for instance: Roselaar S E, Daugherty A “Apolipoprotein E-deficient mice have impaired innate immune responses to listeria monocytogenes in vivo” J Lipid Res 1998, 39:1740-1743.

Potential Uses of ApoE Inducers in the Treatment of Human Diseases (i) In Atherosclerosis (Role of Plasma HDL ApoE)

Increasing ApoE plasma levels will decrease plasma atherogenic lipoproteins (VLDL, IDL and LDL) by increasing their uptake by the liver. Increasing ApoE in HDL will increase the removal of cholesterol from loaded tissues (atherosclerotic arteries) by the reverse cholesterol transport mechanism.

(ii) In the Central Nervous System (CNS)

Increasing ApoE in the brain will prevent the deposition of plaques associated with Alzheimer's disease and increase the repair mechanism of brain injuries due to mechanical traumas or strokes. Through the increase of neurite extension synaptic sprouting the overall brain activity (i.e. memory) should improve.

(iii) In the Peripheral Nervous System (PNS)

ApoE plays an important role in nerve regeneration and increasing ApoE in traumatised nerves (nerve section, crush etc) or degenerative nerves (multiple sclerosis) will increase the speed of the healing process or prevent degeneration.

(iv) In skin Diseases Due to Disturbances in Skin Lipid Metabolism

The skin constitutes a lipophilic barrier and lipid homeostasis is well controlled in cells such as keratinocytes. Cholesterol deposition (xanthelasma and xanthomas) will be prevented by increasing the level of ApoE in skin tissue.

(v) As Modulators of the Immune System

ApoE affects the immune system by acting on lymphocyte proliferation. Furthermore ApoE knock out mice are highly sensitive to bacterial infection due to a defect in innate immune system suggesting that increasing ApoE production should ameliorate the immune response.

For the above reasons the development of compounds which increase ApoE prodution might be useful to treat numerous disease states. Until now only hormones have been shown to affect ApoE synthesis and secretion. The discovery of new small molecules with ApoE modulating activities should lead to the discovery of drugs having application in the treatment of diseases such as:
atherosclerosis,
excess lipid deposition in peripheral tissues such as skin (xanthomas),
stroke,
memory loss,
optic nerve and retinal pathologies (i.e. macular degeneration, retinitis pigmentosa),
repair of traumatic damage of the central nervous system (brain tissue),
repair of traumatic damage of the peripheral nervous system (i.e. nerve section compression or crush),
prevention of the degenerative process due to aging (i.e. Alzheimer's disease),
prevention of degenerative neuropathies occuring in diseases such as diabetic neuropathies and multiple sclerosis,
autoimmune diseases and
activation of the innate immune system.

The applicants have now found that certain FXR and LXRα activators increase ApoE production. Furthermore, the applicants have found that a screening system comprising an in vitro assay allows the rapid identification of ApoE modulators. This in vitro assay involves testing the effects of FXR and LXRα activators on the secretion of ApoE in the THP-1 cell line.

Activators of FXR can be identified by means of the assays described by Forman et al. Cell 1995, 81, 687-693, Makishima, M. et al Science 1999, 284, 1362-1365, Parks, D. J. et al Science 1999, 284, 1365-1368 and Wang, H. B. et al Molecular Cell 1999, 3, 543-553.

Activators of LXRα can be identified by means of the assays described by Willy et al Genes & Development 1995, 9, 1033-1045 and Janowsky B. A. et al. Proc Natl. Acad Sci USA 1999, 96, 266-271.

The present invention is-based on the finding by the inventors that activators of the orphan nuclear receptors FXR such as farnesol, chenodeoxycholic acid and activators of the orphan nuclear receptor LXRα such as 22(R)-hydroxycholesterol, are ApoE modulators and more specifically are ApoE inducers and thus are potentially useful as agents for the treatment of atherosclerotic or neurological diseases such as those previously discussed.

Accordingly, in one aspect, the invention provides a method for increasing Apolipoprotein E (ApoE) in plasma and in tissues of a mammal by using a combination of an ApoE increasing amount of an activator of the orphan nuclear receptor FXR and an ApoE increasing amount of an activator of the orphan nuclear receptor LXRα.

In another aspect, the invention provides a method for increasing ApoE in plasma and in tissues of a mammal by using an ApoE increasing amount of a FXR activator.

In a further aspect, the invention provides a method for increasing ApoE in plasma and in tissues of a mammal by using an ApoE increasing amount of a LXRα activator.

The invention also provides the use of a combination of an ApoE increasing amount of a FXR activator and of an ApoE increasing amount of a LXRα activator, for the manufacture of a medicament for increasing ApoE in plasma and in tissues of a mammal.

In another aspect, the invention provides the use of a FXR activator for the manufacture of a medicament for increasing ApoE in plasma and in tissues of a mammal.

In yet another aspect, the invention provides the use of a LXRα activator for the manufacture of a medicament for increasing ApoE in plasma and in tissues of a mammal.

Moreover the invention also provides the above-mentioned activators, farnesol, chenodeoxycholic acid and 22(R)-hydroxycholesterol for use in medicine, for example for use in therapy, e.g. for use in the treatment or prophylaxis of a disease or condition as hereinbefore defined, and as set out in the claims appended hereto. The invention also provides the use of FXR and LXRα activators for the manufacture of a medicament for the treatment or prophylaxis of a disease or condition as hereinbefore defined or as set out in the claims appended hereto.

The invention further provides a method of treatment or prophylaxis of a disease state or condition as hereinbefore defined, or as set out in the claims appended hereto, which method comprises administering to a subject (e.g. a mammal such as a human) in need thereof, a therapeutically or prophylactically effective (and preferably non-toxic) amount of the FXR activator or the LXRα activator. A further method of treatment of the above-mentioned disease state or condition comprises administering to said subject a combination of an effective amount of the FXR activator and an effective amount of the LXRα activator, by taking advantage of the synergistic effect of the FXR and LXRα activators.

In a further aspect, the invention provides a pharmaceutical composition having apoE increasing activity and comprising both an FXR activator and a LXRα activator. The composition can contain, for example, a mixture of the FXR activator and LXRα activator. The apoE increasing activity of a composition containing both activators may usefully exhibit an apoE increasing activity which is greater than the sum of the apoE increasing activities of the individual activators, i.e. may exhibit synergy. Such synergistic mixtures, compositions containing them and their uses form a further aspect of the invention.

The farnesoid X activated receptor (FXR; NR1H4) is activated by natural compounds from two different chemical classes which comprise certain isoprenoids and certain bile acids.

The isoprenoids that are FXR activators include the following: farnesol, farnesal, farnesyl acetate, farnesoic acid and its esters and juvenile hormone III. It is important to note that not all farnesoids are FXR activators: for instance geranylgeraniol does not activate FXR.

The majority of bile acids are FXR, activators, chenodeoxycholic acid being one of the most potent activators. Examples of bile acid compounds that are not FXR activators are: cholic acid and ursodeoxycholic acid.

Whether or not a compound is a FXR activator can be determined by the assays described by Forman et al. Cell 1995, 81, 687-693, Makishima, M. et al Science 1999, 284, 1362-1365, Parks, D. J. et al Science 1999, 284, 1365-1368 and Wang, H. B. et al Molecular Cell 1999, 3, 543-553.

The currently preferred FXR activators are:
farnesol (trans,trans-farnesol; [2E,6E]-3,7,11-trimethyl-2,6,10-dodecatrien-1-ol)
methyl farnesyl ether, (methyl [2E,6E]-3,7,11-trimethyl-2,6,10-dodecatrien-1-yl ether)
ethyl farnesyl ether, ethyl [2E,6E]-3,7,11-trimethyl-2,6,10-dodecatrien-1-yl ether)
methyl farnesoate, ([2E,6E]-3,7,11-trimethyl-2,6,10-dodecatrienoic acid methyl ester),
ethyl farnesoate, ([2E,6E]-3,7,11-trimethyl-2,6,10-dodecatrienoic acid ethyl ester),
juvenile hormone III (7-methyl-9-(3,3-dimethyloxiranyl)-3-methyl-2,6-nonadienoic acid methyl ester),
7-methyl-9-(3,3-dimethyloxiranyl)-3-methyl-2,6-nonadienoic acid ethyl ester and
chenodeoxycholic acid (3α,7α-dihydroxy-5β-cholanic acid).

The currently most preferred FXR activators are:
farnesol (trans,trans-farnesol; [2E,6E]-3,7,11-trimethyl-2,6,10-dodecatrien-ol)
juvenile hormone III (7-methyl-9-(3,3-dimethyloxiranyl)-3-methyl-2,6-nonadienoic acid methyl ester) and
chenodeoxycholic acid (3α,7α-dihydroxy-5β-cholanic acid).

The Liver X alpha receptor (LXRα, NR1H3) is activated by certain oxysterols which comprise the following: 22(R)-hydroxycholesterol, 24(S)-hydroxycholesterol, 24(S),25-epoxycholesterol, 5,6-24-(S)diepoxycholesterol and 7-hydroxycholesterol. It is again important to note that oxysterols are not all potent activators of LXRα, for instance 22(S)-hydroxycholesterol (the enantiomer of the activator 22(R)-hydroxycholesterol) is a less potent activator of LXRα. Whether or not a compound is a LXRα activator can be determined by the assay described by Willy et al Genes & Development 1995, 9, 1033-1045 and Janowsky B. A. et al Proc Natl Acad Sci USA 1999, 96, 266-271.

The currently preferred LXRα activators are:
22(R)-hydroxycholesterol (5-cholestene-3β,22[R]-diol)
24(S)-hydroxycholesterol,
24(S),25-epoxycholesterol,
5,6-24-(S)diepoxycholesterol and
7-hydroxycholesterol

The currently most preferred LXRα activators are:
22(R)-hydroxycholesterol (5-cholestene-3β,22[R]-diol)
24(S)-hydroxycholesterol and
24(S),25-epoxycholesterol.

This invention arises from the finding by the inventors that ligands and activators of FXR and LXRα are potent inducers of ApoE secretion and might be useful in the treatment of diseases requiring an increased production of plasma and/or tissue ApoE described above. The ApoE inducing activity of these compounds was demonstrated in an in vitro model, the ApoE-producing cell line THP-1, where a representative member of each chemical class of activators was tested in parallel with a closely related compound which is not an activator of the specific nuclear receptor. The test results show that the activators are several-fold more potent in inducing ApoE than their non-inducer analogs, thereby establishing the potency and specificity of the activity of the FXR and LXRα activators.

In a further aspect, the invention provides a method for identifying a candidate compound for use in increasing apolipoprotein E (ApoE) levels and tissues and plasma of a mammal; which method comprises selecting a test compound having FXR nuclear receptor activator activity and thereafter subjecting the test compound to an assay to determine its apoE inducing effect.

In another aspect the invention provides a method for identifying a candidate compound for use in increasing apolipoprotein E (ApoE) levels and tissues and plasma of a mammal; which method comprises selecting a test compound having LXRα nuclear receptor activator activity and thereafter subjecting the test compound to an assay to determine its ApoE inducing effect.

Each of the above methods may further comprise a preliminary step of subjecting a test compound to an assay to determine whether the compound is a FXR or LXRα activator respectively.

The aforementioned methods may optionally comprise formulating a candidate compound identified by the method as a pharmaceutical formulation as a medicament.

The medicaments prepared according to the foregoing methods may be used for any of the therapeutic purposes disclosed herein for apoE modulator compounds.

The FXR or the LXRα activator of the invention can be administered by any of a variety of routes. Thus, for example, they can be administered orally, or by delivery across another mucosal surface (for example across the nasal, buccal, bronchial or rectal mucosa), transdermally, or by injection (for example intradermal, intraperitoneal, intravenous or intramuscular injection).

When the compounds are intended for oral administration, they can be formulated, for example, as tablets, capsules, granules, pills, lozenges, powders, solutions, emulsions, syrups, suspensions, or any other pharmaceutical form suitable for oral administration. Oral dosage forms can, if desired, be coated with one or more release delaying coatings to allow the release of the active compound to be controlled or targeted at a particular part of the enteric tract.

Tablets and other solid or liquid oral dosage forms can be prepared (e.g. in standard fashion) from the FXR or the LXRα activator and a pharmaceutically acceptable solubilizer, diluent or carrier. Examples of solubilizers, diluents or carriers include sugars such as lactose, starches, cellulose and its derivatives, powdered tracaganth, malt, gelatin, talc, stearic acid, magnesium stearate, calcium sulfate, vegetable oils, polyols such as glycerol, propyleneglycol and polyethyleneglycols, alginic acids and alginates, agar, pyrogen free water, isotonic. saline, phosphate buffered solutions, and optionally other pharmaceutical excipients such as disintegrants, lubricants, wetting agents such as sodium lauryl sulfate, coloring agents, flavoring agents and preservatives, etc.

Capsules can be of the hard or soft variety and can contain the active compound in solid, liquid or semisolid form. Typically such capsules are formed from gelatine or an equivalent substance and can be coated or uncoated. If it is desired to delay the release of the active compound until the capsule has passed through the stomach and into the intestine, the capsule can be provided with a pH sensitive coating adapted to dissolve at the pH found in the duodenum or ileum. Examples of such coatings include the Eudragits, the uses of which are well known.

Formulations for injection will usually be made up of the appropriate, solubilizers such as detergents which may also include compounds and excipients such as buffering agents to provide an isotonic solution having the correct physiological pH. The injectable solutions are typically pyrogen-free and can be provided in sealed vials or ampoules containing a unit dose of compound.

A unit dosage form of the compounds of the invention typically will contain from 0.1% to 99% by weight of the active substance, more usually from 5% to 75% of the active substance. By way of example, a unit dosage form can contain from 1 mg to 1 g of the compound, more usually from 10 mg to 500 mg, for example between 50 mg and 400 mg, and typically in doses of 100 mg to 200 mg.

The compounds of the invention will be administered in amounts which are effective to provide the desired therapeutic effect. The concentrations necessary to provide the desired therapeutic effect will vary according to among other things the precise nature of the disease, the size, weight and age of the patient and the severity of the disease.

The doses. administered will preferably be non-toxic to the patient, although in certain circumstances the severity of the disease under treatment may necessitate administering an amount of compound which causes some signs of toxicity.

Typically, the compounds of the invention will be administered in amounts in the range 0.01 mg/kg to 100 mg/kg body weight, more preferably 0.1 mg/kg to 10 mg/kg body weight and particularly 1 mg/kg to 5 mg/kg body weight. For an average human of 70 kg weight, a typical daily dosage of the compounds of the invention would be in the range of 70 mg to 700 mg. Such a dosage can be administered, for example from two to four times daily. Ultimately however, the size of the doses administered and the frequency of administration will be at the discretion and judgement of the physician treating the patient.

For therapeutic use the. compounds of the present invention will generally be administered in a standard pharmaceutical composition obtained by admixture with a pharmaceutical carrier selected with regard to the intended route of administration and standard pharmaceutical practice. For example, they may be administered orally in the form of tablets containing such excipients as starch or lactose, or in capsule, ovules or lozenges either alone or in admixture with excipients, or in the form of elixirs or suspensions containing flavoring or coloring agents. They may be injected parenterally, for example, intravenously, intramuscularly or subcutaneously. For parenteral administration, they are best used in the form of a sterile aqueous solution which may contain other substances, for example, enough salts or glucose to make the solution isotonic with blood. The choice of form for administration as well as effective dosages will vary depending, inter alia, on the condition being treated. The choice of mode of administration and dosage is within the skill of the art.

The FXR or LXRα activators and their pharmaceutically acceptable salts which are active when given orally can be formulated as liquids, for example syrups, suspensions or emulsions or as solids for example, tablets, capsules and lozenges. A liquid formulation will generally consist of a suspension or solution of the compound or pharmaceutically acceptable salt in suitable liquid carrier(s) for example, ethanol, glycerine, non-aqueous solvent, for example polyethylene glycol, oils, or water with a suspending agent, preservative, flavoring or coloring agents. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatine capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

A typical suppository formulation comprises the FXR or LXRα activator or a pharmaceutically acceptable salt thereof which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule.

Each dosage unit for oral administration contains preferably from 1 to 250 mg (and for parenteral administration contains preferably from 0.1 to 25 mg) of the FXR or LXRα activator or a pharmaceutically acceptable salt thereof calculated as the free base.

The pharmaceutically acceptable compounds of the invention will normally be administered to a subject in a daily dosage regimen. For an adult patient this may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the FXR or LXRα activator or a pharmaceutically acceptable salt thereof calculated as the free base, the compound being administered 1 to 4 times per day.

Disease states which could benefit from increasing plasma and tissue ApoE levels include, but are not limited to: atherosclerosis, neurodegenerative disorders such as Alzheimer's disease or dementia. The compounds of this invention modulate ApoE and are therefore of value in the treatment of any of these conditions.

### DETAILED DESCRIPTION OF THE PREFERRED EMBODIMENTS

The invention will be now illustrated, but not limited, by reference to the following examples.

### EXAMPLE 1

A. Method of Determining Biological Activity:

The FXR and LXRα activators of the invention increase the ApoE production in vitro. The activities of the compounds can be determined an in vitro assay which comprises testing the effects of FXR and LXRα activators on the secretion of ApoE in the THP-1 cell line. Previous studies performed by the applicants had shown that test compounds which induce the secretion of ApoE by the ApoE-secreting cell line (THP-1 cells), are active in vivo for increasing plasma and tissue ApoE when given to animals from a wide range of species.

B. Cell Culture

The THP1 cell line was derived from the peripheral blood of a 1-year-old boy with acute monocytic leukemia. These cells did not express surface and cytoplasmic immunoglobulins; they were phagocytic and differentiated into macrophage-like cells. These cells were obtained from the European Collection of Animal Cell Cultures (ECACC, #88081201). The cells were grown as non-adherent cells in RPMI 1640 culture medium, 2 mM glutamine, 20 μM 2-mercaptoethanol. Fresh medium was added to maintain cell density between 2 and 9×10⁵ cells/ml. Once a week, new cultures were initiated by inoculating 10 ml of medium with 2×10⁶ cells in a 75 cm² plate. The plates were kept at 37° C. in a 5% CO₂ atmosphere. For screening, cells were seeded in 24-well plates at the density of 2×10⁵ cells per well. Phorbol-12-myristat-13-acetate (PMA) was added at 0 and 2.5 nM to initiate THP1 differentiation into adherent macrophage-like cells. Vehicles, reference compounds and test compounds were added simultaneously at concentrations varying from 1 to 50 μM and incubated for 72 hours. The culture medium was then recovered, centrifuged at 300 g for 5 min to remove any unattached cell and stored at −20° C. before analysis.

C. Apo E Determination by ELISA

96 well-microtiter plates were coated by incubating with a 5% gelatine solution from porcine skin, in 50 mM carbonate-bicarbonate buffer, pH9.6 at the concentration of 200 μl/well, for 2 hours at 37° C. The coating solution was carefully removed and the test compound was added (100 μl/well) at the appropriate dilution. Dilutions of a human ApoE standard (ICN BiomedicalsInc, USA) were simultaneously assayed. Test compounds and antibodies were diluted in the following buffer solution: PBS, 1% BSA, 0.1% Tween 20, pH 7.4. Test compounds were incubated for 1 hour at 37° C., and the wells were washed 3 times with 200 μl of buffer solution. One hundred microliters per well of the primary antibody (goat anti-human ApoE IgG) diluted 10000 fold was incubated for 1 hour at 37° C. under continuous shaking. After the third wash, 100 μl/well of the secondary antibody (anti-goat-IgG peroxidase conjugate) diluted 5000 fold was incubated for 1 hour at 37° C. with continuous shaking. Wells were washed 5 times and 100 μl/well of substrate (ortho-phenylenediamine dihydrochloride) was incubated for the appropriate time at room temperature in the dark with continuous shaking. The reaction was stopped by adding 50 μl/well of 3M sulfuric acid and incubating for 1 min at room temperature. The absorbance at 492 run versus 620 nm was read on a microplate photometer and the results were then converted into human ApoE ng equivalent.

D. ApoE Inducing Activity of Farnesol, Chenoxycholic Acid and 22(R)-Hydroxycholesterol

Representative examples of FXR and LXRα activators were tested for their activity in modulating the secretion of ApoE by THP-1 cells in the conditions described above and the results obtained are reported in Tables 1, 2 and 3. In each instance the FXR or the LXRα activator, was tested at the same concentration as that of a close analog from the same chemical class which is not an activator, or is a much weaker activator, of the nuclear receptor. This experimental design was devised to demonstrate the potency and selectivity of the FXR and LXRα activators.

Thus, the ApoE inducing effect of two different chemical classes of FXR activator is demonstrated in Table 1 and 2. Farnesol, a representative example of FXR activator selected from the isoprenoid class, increases ApoE secretion by 125% whereas the inactive isoprenoid geranylgeraniol increases ApoE by only 36% (Table 1). Likewise, the bile acid derivative chenodeoxycholic acid, a well characterised ligand and activator of FXR, increased ApoE production by 138% while the close analog, cholic acid which does not activate FXR, did not change ApoE secretion by THP-1 cells (Table 2).

The direct involvement of the orphan nuclear receptor LXRα in the induction of ApoE is clearly demonstrated by the fact that the activator enantiomer, 22(R)-hydroxycholesterol increases ApoE secretion by 250% whereas the much weaker enantiomer, 22(S)-hydroxycholesterol, was inactive: +23% at the same concentration (Table 3). In particular, the potency of 22(R)-hydroxycholesterol should be noted since significant increases in ApoE secretion could already be measured at submicromolar concentrations.

All the compounds tested were commercially available and were purchased from Aldrich (Farnesol, # 27,754-1) or Sigma (Geranylgeraniol, # G3278, Chenodeoxycholic acid, # C9377, Cholic acid, # C1129, 22(R)-hydroxycholesterol, # H9384 and 22(S)-hydroxycholesterol, # H5884).

TABLE 1
Chemical Class: Isoprenoid

| Compound | Interaction with | Change in Apo E |
|---|---|---|
| (20 μM) | nuclear receptor | production |
| Farnesol | FXR activator ¹⁾ | +125% |
| (Trans,trans-Farnesol; | | |
| [2E,6E]-3,7,11-trimethyl- | | |
| 2,6,10-dodecatrien-ol) | | |
| Geranylgeraniol | Does not activate | +36% |
| (All trans-3,7,11,15-tetra- | FXR ¹⁾ | |
| methyl-2,6,10,14-hexadeca- | | |
| tetraen-1-ol) | | |

¹⁾ See for instance: Forman et al. Cell 1995, 81, 687-693

TABLE 2
Chemical Class: Bile Acid

| Compound | Interaction with | Change in Apo E |
|---|---|---|
| (50 μM) | Nuclear receptor | production |
| Chenodeoxycholic acid | FXR activator ²⁾ | +138% |
| (3α,7α-dihydroxy-5β- | | |
| cholanic acid) | | |
| Cholic acid | Does not activate | +6% |
| (3α,7α,12α-trihydroxy- | FXR ²⁾ | |
| 5β-cholan-24-oic acid) | | |

²⁾ See for instance: Makishima, M. et al Science 1999, 284, 1362-1365. Parks, D. J. et al Science 1999, 284, 1365-1368. Wang, H. B. et al Molecular Cell 1999, 3, 543-553.

TABLE 3
Chemical Class: Hydroxy Sterol

| Compound | Interaction with | Change in Apo E |
|---|---|---|
| (1 μM) | Nuclear receptor | production |
| 22(R)-hydroxycholesterol | LXRα activator ³⁾ | +249% |
| (5-cholestene-3β,22[R]-diol) | | |
| 22(S)-hydroxycholesterol | Less potent activator | +23% |
| (5-cholestene-3β,22[S]-diol) | of LXRα ³⁾ | |

³⁾ See for instance: Janowsky B. A. et al Proc Natl Acad Sci USA 1999, 96, 266-271

The foregoing examples are intended merely to be illustrative of the invention and are not intended to limit the scope of the invention in any way. It will readily be apparent that numerous modifications and alterations may be made to the examples without departing from the principles underlying the invention and all such modifications and alterations are intended to be embraced by this application.

1. A method for increasing apolipoprotein E (ApoE) in plasma and in tissues of a mammal comprising administering to said mammal an ApoE increasing amount of an activator of the orphan nuclear receptor FXR and an ApoE increasing amount of an activator of the orphan nuclear receptor LXRα.

2. A method for increasing ApoE in plasma and in tissues of a mammal comprising admininstering to said mammal an ApoE increasing amount of a FXR activator.

3. A method for increasing ApoE in plasma and in tissues of a mammal comprising administering to said mammal an ApoE increasing amount of a LXRα activator.

7. A method of regulating regulating cholesterol homeostasis in a mammal comprising administering to said mammal at least one of a FXR activator or a LXRα activator.

8. The method of claim 1, wherein the FXR activator is an isoprenoid or a bile acid.

9. The method of claim 8, wherein the activator of the orphan nuclear receptor FXR is selected from:
farrnesol (trans,trans-farnesol;-3,7,11-trimethyl-2,6,10dodecatrien-1-ol),
methyl farnesyl ether, (methyl-3,7,11-trimethyl-2,6,10dodecatrien-1-yl ether),
ethyl farnesyl ether, ethyl-3,7,11-trimethyl-2,6,10dodecatrien-1-yl ether),
methyl farnesoate, (-3,7,11-tnmethyl-2,6,10-dodecatrienoic acid methyl ester),
ethyl farnesoate, (-3,7,11-trimethyl-2,6,10-dodecatrienoic acid ethyl ester),
juvenile hormone III (7-methyl-9-(3,3-dimethyloxiranyl)-3-methyl2,6-nonadienoic acid methyl ester),
7-methyl-9-(3,3-dimethyloxiranyl)-3-methyl-2,6-nonadienoic acid ethyl ester and
chenodeoxycholic acid (3a,7a-dihydroxy-5(3-cholanic acid).

10. The method of claim 9, wherein the activator of the orphan nuclear receptor FXR is selected from:
farnesol (trans,trans-fanesol;-3,7,11-trimethyl-2,6,10dodecatrien-1-ol),
juvenile hormone III (7-methyl-9-(3,3-dimethyloxiranyl)-3-methyl2,6-nonadienoic acid methyl ester) and
chenodeoxycholic acid (3a,7(x-dihydroxy-5(3-cholanic acid).

11. The method of claim 1, wherein the activator of the orphan nuclear receptor LXRα is a cholesterol derivative.

12. The method of claim 11, wherein the cholesterol derivative is selected from:
22(R)-hydroxycholesterol (5-cholestene-3(3,22-diol)
24(S)-hydroxycholesterol,
24(5),25-epoxycholesterol,
5,6-24-(S)diepoxycholesterol and
7-hydroxycholesterol.

13. The method of claim 12, wherein the activator of the orphan nuclear receptor LXRα is 22(R)-hydroxycholesterol (5-cholestene-3β,22-diol).

14. A pharmaceutical composition having apoE increasing activity and comprising both an FXR activator and a LXRα activator in a pharmaceutically acceptable excipient.

15. The pharmaceutical composition of claim 14, wherein the FXR activator is an isoprenoid or a bile acid.

16. The pharmaceutical composition of claim 15, wherein the activator of the orphan nuclear receptor FXR is selected from:
farrnesol (trans,trans-farnesol;-3,7,11-trimethyl-2,6,10dodecatrien-1-ol),
methyl farnesyl ether, (methyl-3,7,11-trimethyl-2,6,10dodecatrien-1-yl ether),
ethyl farnesyl ether, ethyl -3,7,11-trimethyl-2,6,10dodecatrien-1-yl ether),
methyl farnesoate, (-3,7,11-tnmethyl-2,6,10-dodecatrienoic acid methyl ester),
ethyl farnesoate, (-3,7,11-trimethyl-2,6,10-dodecatrienoic acid ethyl ester),
juvenile hormone III (7-methyl-9-(3,3-dimethyloxiranyl)-3-methyl2,6-nonadienoic acid methyl ester),
7-methyl-9-(3,3-dimethyloxiranyl)-3-methyl-2,6-nonadienoic acid ethyl ester and
chenodeoxycholic acid (3a,7a-dihydroxy-5(3-cholanic acid).

17. The method of claim 1, wherein said mammal has atherosclerosis.

18. The pharmaceutical composition of claim 14, wherein the activator of the orphan nuclear receptor LXRα is a cholesterol derivative.

19. The method of claim 1, wherein said mammal has macular degeneration and retinitis pigmentosa.

20. The method of claim 1, wherein said mammal has suffered a stroke.

21. The method of claim 1, wherein said mammal has a degenerative neuropathy, such as diabetic neuropathies or multiple sclerosis.

22. The method of claim 1, wherein said mammal has Alzheimer's disease or dementia.

23. The method of claim 25, wherein said method comprises the combination of an in vitro assay and an in vivo animal model assay, said in vitro assay comprising testing said candidate compound for activity in inhibiting or inducing the secretion of ApoE in the THP-1 cell line, and said in vivo animal model assay comprising testing said candidate compound in a rat for a modulating effect on plasma ApoE.

24. A method of screening a candidate compound for therapeutic use as an ApoE modulator comprising selecting a compound and screening said candidate compound for FXR or LXRα activator activity wherein FXR or LXRα activator activity identifies the candidate compound as an ApoE modulator.

25. The method of claim 24, further comprising the step of assessing the ApoE modulatory activity of said candidate compound.

26. The method of claim 24, further comprising the step of formulating said compound to form a pharmaceutical composition.

27. The pharmaceutical composition of claim 18, wherein the cholesterol derivative is selected from:
22(R)-hydroxycholesterol (5-cholestene-3(3,22-diol)
24(S)-hydroxycholesterol,
24(5),25-epoxycholesterol,
5,6-24-(S)diepoxycholesterol and
7-hydroxycholesterol.
